(19) United States
(12) Patent Application Publication
(10) Pub. No.: US 2012/0250823 A1
(43) Pub. Date: Oct. 4, 2012

(54) CALIBRATION OF DIFFERENTIAL PHASE-CONTRAST IMAGING SYSTEMS

(75) Inventors: Gereon Vogtmeier, Aachen (DE); Klaus Juergen Engel, Aachen (DE); Dieter Geller, Aachen (DE); Thomas Koehler, Norderstedt (DE); Ewald Roessl, Ellerau (DE)

(73) Assignee: KONINKLIJKE PHILIPS ELECTRONICS N.V., EINDHOVEN (NL)

(21) Appl. No.: 13/514,166

(22) PCT Filed: Dec. 8, 2010
(86) PCT No.: PCT/IB10/55664

§ 371 Date: Jun. 6, 2012

(30) Foreign Application Priority Data
Dec. 10, 2009 (EP) 09178691.3

(51) Int. Cl. A61B 6/02

(52) U.S. Cl. 378/62

(57) ABSTRACT

The present invention relates to an X-ray imaging system and a method for differential phase—contrast imaging of an object. To improve calibration of differential phase—contrast imaging systems and the alignment of the gratings an X-ray imaging system is provided that comprises an X-ray emitting arrangement providing at least partially coherent X-ray radiation and an X-ray detection arrangement comprising a phase-shift diffraction grating, a phase analyzer grating, and an X-ray image detector, all arranged along an optical axis. For stepping, the gratings and/or the X-ray emitting arrangement are provided with at least two actuators arranged opposite to each other with reference to the optical axis. For calibration, calibration projections are acquired without an object, wherein, the emitted X-ray radiation or one of the gratings is stepwise displaced with a calibration displacement value. For examination, measurement projections are acquired with an object, wherein the emitted X-ray radiation or one of the gratings is stepwise displaced with a measurement, a calibration projection is associated to each of the measurement projections by registering the latter with the calibration projections.

### FIELD OF THE INVENTION

The present invention relates to an X-ray imaging system for differential phase-contrast imaging of an object and a method for acquiring information about an object based on differential phase-contrast imaging.

### BACKGROUND OF THE INVENTION

X-ray differential phase-contrast imaging (DPCI) visualizes the phase information of coherent X-rays passing a scanned object. In addition to classical X-ray transmission imaging, DPCI determines not only the absorption properties of a scanned object along a projection line, but also the phase-shift of the transmitted X-ray, and thus provides valuable additional information usable for contrast enhancement, material composition or dose reduction, to name a few examples. Independent whether a coherent X-ray source is used or a standard X-ray source with an additional source grating, as described in EP 1 731 099 A1, which assures coherence through small openings, a phase-shift grating is placed after the object, working as a beam splitter. The resulting interference pattern contains the required information about the beam phase-shift in the relative positions of its minima and maxima, typically in the order of several micrometers. Since a common X-ray detector, typical resolution in the order 150 μm, is not able to resolve such fine structures, the interference is sampled with a phase analyzer grating, also known as absorber grating. The phase analyzer grating features a periodic pattern of transmitting and absorbing strips with the periodicity similar to that of the interference pattern. The similar periodicity produces a Moiré pattern behind the grating with a much larger periodicity, which is detectable by common X-ray detectors. To obtain the phase-shift, a shifting of one of the gratings laterally by fractions of the grating pitch is provided, for which also the term phase stepping is used. The phase-shift can be extracted from the particular Moiré pattern measured for each position of the analyzer grating. It has been shown that the setup with different gratings requires a good calibration for acquisition of reliable data. This is even more severe in a larger system that might consist of several tiles of gratings and detectors which will be placed like a mosaic to have a large effective detection area, for example. For the setup with linear gratings, the parallel alignment of the gratings is important, as even small deviations of a parallel alignment produce additional fringes in the detective Moiré pattern which aggravate an accurate image analysis and make the system more sensitive to mechanical instabilities.

### SUMMARY OF THE INVENTION

Hence, there may be a need to improve calibration of differential phase-contrast imaging systems and the alignment of the gratings provided in differential phase-contrast imaging systems.

According to an exemplary embodiment, a method for acquisition of information about an object is provided that comprises the following steps: a) Emitting at least partially coherent X-ray radiation from an X-ray emitting arrangement towards an X-ray detection arrangement, wherein the X-ray detection arrangement comprises a phase-shift diffraction grating, a phase analyzer grating and an X-ray image detector, wherein the X-ray emitting arrangement, the phase-shift grating, the phase analyzer grating and the image detector are arranged along an optical axis, and wherein the emitted at least partially coherent X-ray radiation, the phase-shift grating and the phase analyzer grating have a common grid orientation; b) Performing a first plurality of calibration projections without an object, wherein, during the first plurality of calibration projections, the emitted X-ray radiation or one of the group of the phase-shift grating and the phase analyzer grating is stepwise displaced with a calibration displacement value; c) Performing a second plurality of measurement projections with an object arranged between the X-ray emitting arrangement and the phase analyzer grating, wherein, during the second plurality of measurement projections, the emitted X-ray radiation or one of the group of the phase-shift grating and the phase analyzer grating is stepwise displaced with a measurement increment; and d) associating at least one of the calibration projections to each of the measurement projections by registering the measurement projections with the calibration projections.

According to an exemplary embodiment, in order to register the calibration projection with the measurement projection, the measurement projection is analyzed for parts which are illuminated directly. Depending on the actual position of the gratings, for example due to translation, rotation, tilt or the like, a characteristic fringe pattern is visible in these areas. In the second step of the registration process, the projection from the plurality of the calibration projections is identified which shows in the same area the most similar fringe pattern.

According to an exemplary embodiment, during the second plurality of measurement projections, the object is arranged between the X-ray emitting arrangement and the phase-shift diffraction grating, such that a region of interest of the object is exposable to the X-ray radiation emitting from the X-ray emitting arrangement towards the detector.

According to another exemplary embodiment, during the second plurality of measurement projections, the object is arranged between the X-ray emitting arrangement and the phase analyzer grating, or, in other words, between phase-shift grating and the analyzer grating, i.e. in direction of the X-ray beams behind the phase-shift grating, such that a region of interest of the object is exposable to the X-ray radiation emitting from the X-ray emitting arrangement towards the detector.

According to an exemplary embodiment, after the step d) the following steps are performed: e) Generating adjusted measurement projections by subtracting the respective associated calibration scan from each of the measurement projections; f) determining differential phase data from the adjusted measurement projections; g) generating object information on behalf of the determined differential phase data.

According to an exemplary embodiment, after the step g) the object information is provided, for example for further steps.

According to an exemplary embodiment, the object information is provided to the user, for example by displaying the object information.

According to an exemplary embodiment, the displacement comprises translation, rotation, and tilting of the gratings.

The term “stepwise displacement” comprises a one-dimensional movement as well as a two- or more-dimensional movement, e.g. a three-dimensional movement track in space.

Thus it is possible to create a multidimensional parameter space, or multidimensional movement space. Thereby, the calibration projections can be adapted to different possible misalignments.

According to an exemplary embodiment, the displacement value is a predetermined factor with same value for each step.

Alternatively, the displacement value changes constantly, for example by a constant mathematical function or by predetermined fixed values.

The term “stepwise displacement” may also comprise a continuing movement provided that with respect to each projection no measureable relative movement between the X-ray source and the detector occur. This is the case, for example, during relatively slow movement and short exposure times for each of the projections.

For example, stepwise displacement, or scanning, is provided in fine steps in a linear direction perpendicular to the optical axis and in the same time, rotation around the optical axis is realized representing rotation between the X-ray emitting arrangement and the phase-shift grating or the phase analyzer grating.

It is noted that the “phase analyzer grating” is also referred to as “analyzer grating”. Further, the X-ray image detector is also referred to as X-ray imaging detector.

According to an exemplary embodiment, the phase-shift grating and the phase analyzer grating are arranged in planes parallel to each other.

According to an exemplary embodiment, the calibration displacement value differs from the measurement increment.

According to an exemplary embodiment, the number of the first plurality of calibration projections is at least twice as high as the number of the second plurality of measurement projections.

This provides the advantage that the calibration projections can be acquired independent of the object at an earlier time. For example, in case the object is a patient, the calibration projections can be acquired before, which reduces the necessary time the patient has to be present in the examination apparatus. The invention further provides the advantage that even if the patient scanning leads to a misalignment, a precise detection and thus precise data generation is ensured. For example, in case the examination procedure is a breast cancer examination, the arrangement of the breast between two holding devices often results in tilting or twisting forces leading to a misalignment within the system. But since the calibration projections have been acquired in a larger number beforehand, it is possible to register a particular measurement scan with a matching calibration scan thus providing calibration possibility for each of the measurement projections. Hence, precise data can be generated, because the invention provides scanning a plurality of calibration projections such that it is ensured that for all misalignments that under normal conditions can be expected, a respective calibration scan is provided.

According to an exemplary embodiment of the invention, an X-ray imaging system for differential phase-contrast imaging of an object is provided comprising an X-ray emitting arrangement and an X-ray detection arrangement. The X-ray emitting arrangement provides at least partially coherent X-ray radiation. The X-ray detection arrangement comprises a phase-shift diffraction grating, a phase analyzer grating and an X-ray image detector. The X-ray emitting arrangement, the phase-shift grating and the phase analyzer grating and the image detector are arranged in this order along an optical axis. An object to be examined is receivable between the X-ray emitting arrangement and the phase analyzer grating such that a region of interest of the object is exposable to X-ray radiation emitting from the X-ray emitting arrangement towards the detector. At least one of the group of one of the gratings and the X-ray emitting arrangement is provided with at least two actuators arranged opposite to each other with reference to the optical axis.

One of the advantages is that the actuators allow a movement of the components of the system in relation to each other.

According to an exemplary embodiment, the X-ray emitting arrangement provides X-ray radiation with at least 20% coherent radiation.

According to another exemplary embodiment, the X-ray emitting arrangement provides X-ray radiation with at least 50% coherent radiation.

According to an exemplary embodiment, the X-ray emitting arrangement provides coherent X-ray radiation.

For example, the X-ray radiation is spatially coherent.

According to an exemplary embodiment, the phase-shift grating and the phase analyzer grating are arranged in planes parallel to each other.

According to an exemplary embodiment, the gratings are rectangular and the actuators are arranged diametrically to each other.

Thereby, a movement of at least one of the gratings is provided that can be controlled due to the positioning of the actuators diametrically to each other.

According to an exemplary embodiment of the invention, the actuators are arranged near the edge of the gratings, for example, to provide good leverage or a good transformation ratio.

Arranging the actuators with a distance to each other allows fine-tuning of the movement whereas an arrangement with actuators located close to each other would mean large transformation or movement of a grating by only a small actuating movement of the actuator.

According to an exemplary embodiment of the invention, the at least two actuators provide movement in a plane perpendicular to the optical axis.

This allows for an alignment of the gratings and the X-ray emitting arrangement respectively, during which alignment the parallel arrangement of the gratings is ensured.

According to an exemplary embodiment of the invention, the at least two actuators provide stepping movement of at least one of the group of one of the gratings and the X-ray emitting arrangement for phase stepping image acquisition and also provide calibration movement for calibrating the system in order to detect and to compensate misalignment of the X-ray emitting arrangement and the phase-shift grating and the phase analyzer grating.

This provides the advantage that the same movement mechanism can be used both for phase stepping and for calibration and alignment. Following, the system can be implemented with less components which provides a facilitated manufacturing process and also provides economic benefits. Further, it is also possible to implement the system requiring less space.

According to an exemplary embodiment of the invention, the at least two actuators each provide linear movement in a direction which is perpendicular to the grid orientation and which is also perpendicular to the optical axis.

According to an exemplary embodiment, the at least two actuators each provide movement in the x-axis such that linear movement of the grating is provided by moving of the actuators with same speed in same direction and such that rotation is provided by moving in different directions.

Thus, although the actuators are provided with the same type of movement, namely linear movement, different movement types of the grating, for example, can be achieved by different controlling of the actuators.

According to an exemplary embodiment, the rotational movement depends on the location and type of the fixture point.

According to an exemplary embodiment, the linear movement is provided for phase scanning and the rotational movement is provided for calibration purposes.

According to an exemplary embodiment, the at least two actuators provide transversal displacement of the grating perpendicular to the optical axis and rotational movement of the grating around the optical axis.

According to an exemplary embodiment of the invention, the at least two actuators are provided at the phase analyzer grating to provide lateral shifting of the grating by fractions of the grating pitch.

According to an exemplary embodiment, the lateral shifting comprises movement perpendicular to the grid orientation and movement perpendicular to the optical axis.

According to an exemplary embodiment, the optical axis is referred to as the z-axis, the grid orientation which is perpendicular to the z-axis is referred to as the y-axis and the axis perpendicular to the grid orientation and perpendicular to the optical axis is referred to as the x-axis.

According to an exemplary embodiment, the at least two actuators form a double actuator.

Hence, the double actuator provides movement in different direction which movement can be combined by the individual movements of the two separate actuators acting together.

According to an exemplary embodiment, a micro-focus tube or a synchrotron-type tube is provided as X-ray radiation source.

For example, carbon nano-tubes are provided to generate at least partial coherent X-ray radiation.

According to a different exemplary embodiment, the X-ray emitting arrangement comprises an X-ray source emitting incoherent X-ray radiation and a source grating is placed close to the X-ray source to provide at least partial spatial beam coherence.

Thus, normal X-ray tubes, for example, can be used.

According to an exemplary embodiment, the at least two actuators are provided at the source grating to provide lateral shifting of the source grating by fractions of the grating pitch.

Thereby, it is possible to move the source grating for the phase stepping and also to move the source grating to provide a correct alignment, for example.

According to an exemplary embodiment, the source grating is an absorbing grating comprising a plurality of transmitting slits in a first pitch, wherein the slits of the source grating create an array of individually coherent, but mutually incoherent sources.

According to an exemplary embodiment, the phase-shift grating features a periodic pattern of transmitting and absorbing strips with a second pitch.

According to an exemplary embodiment of the invention, the phase analyzer grating features a periodic pattern of transmitting and absorbing strips with a third pitch.

According to an exemplary embodiment, the source grating provides an interference pattern between the source grating and the phase-shift grating.

According to an exemplary embodiment, the source grating is laterally shiftable.

The source grating is, for example, shiftable by fractions of the grating pitch of the source grating. Thus, the source grating can be moved to provide the necessary movement for the phase stepping action as well as movement in order to provide correct alignment.

According to an exemplary embodiment, the phase-shift grating is laterally shiftable, for example by fractions of the grating pitch.

According to an exemplary embodiment, the phase analyzer grating is laterally shiftable, for example by fractions of the grating pitch.

By providing one or two or all three of the gratings as being laterally shiftable, an optimum alignment along the optical axis can be achieved by controlling of the respective actuators.

According to an exemplary embodiment, at least two of the gratings are each provided with at least two actuators arranged on the respective grating opposite to each other with reference to the optical axis.

According to an exemplary embodiment, one of the phase-shift grating and the phase analyzer grating is fixedly mounted and the other one is movably mounted. The at least two actuators are provided at the movably mounted grating such that the phase-shift grating and the phase analyzer grating can be aligned in relation to each other.

This reduces the number of necessary components to a minimum in order to provide both phase scanning movement and calibration movement.

According to an exemplary embodiment, the movably mounted grating is movably mounted to the fixedly mounted grating by means of the at least two actuators.

Hence, the same components, namely the actuators, are used for two different purposes which further facilitates the setup of the system.

According to an exemplary embodiment, the source grating is provided with at least two actuators such that it can be aligned and stepwise scanned independently. The phase-shift grating and the phase analyzer grating are movably arranged as a unit.

According to an exemplary embodiment, the at least two actuators are provided as piezo-drive elements with a solid-state hinge.

Piezo-drive elements provide precise and exact movement in the micrometer scale. Piezo-drive elements also provide small and reliable actuators providing even very small amounts of movement.

According to an exemplary embodiment, the actuators are integrally implemented with the grating in silicon by micro-electro-mechanical-systems methods.

According to an exemplary embodiment, at least one additional actuator is provided which actuator is adapted for movement in the direction of the optical axis such that at least one of the gratings can be tilted in relation to the optical axis.

This allows for an alignment of the gratings also in relation to the optical axis.

According to an exemplary embodiment, the at least one additional actuator is adapted such to provide parallel alignment of the gratings in relation to each other.

According to an exemplary embodiment, the grid orientation is perpendicular to the optical axis.

According to an exemplary embodiment, the registration is based on spatial information provided in the calibration projections and in the measurement projections.

According to an exemplary embodiment, the spatial information of the calibration projections is compared with spatial information of the measurement projections and projections with matching spatial information are associated to each other.

According to an exemplary embodiment, the spatial information is provided by predetermined areas scanned outside the object within the calibration projections and within the measurement projections.

According to an exemplary embodiment, the spatial information is provided within free areas of the calibration projections and free areas of the measurement projections.

According to an exemplary embodiment, the X-ray emitting arrangement comprises an X-ray source emitting incoherent X-ray radiation and a source grating is placed close to the X-ray source to provide spatial beam coherence. The source grating is displaced during the calibration projections and during the measurement projections.

According to an exemplary embodiment, the phase-shift grating or the analyzer grating is displaced during the calibration projections and during the measurement projections.

According to an exemplary embodiment, at least one of the group of one the gratings and the X-ray emitting arrangement is provided with at least two actuators arranged at the grating opposite to each other with reference to the optical axis. The at least two actuators provide the displacement during the calibration projections and during the measurement projections.

According to an exemplary embodiment, the calibration stepwise displacement comprises a stepping in a direction perpendicular to the grid orientation.

According to an exemplary embodiment, the calibration stepwise displacement comprises a twisting displacement in relation to the optical axis.

This provides the possibility, to generate different movement sequences.

According to an exemplary embodiment, the phase-shift grating and the phase analyzer grating are fixed in relation to each other.

According to an exemplary embodiment, the number of the first plurality of calibration projections is ten times as high as the numbers of the second plurality of measurement projections.

Thus, it is ensured that surplus or at least enough calibration projections are provided covering possible misalignments.

According to an exemplary embodiment, the calibration displacement value is a constant value.

For example, by adapting the value to be small enough, it is ensured that a fine stepping during the calibration projections is provided.

According to an exemplary embodiment, the calibration displacement value is generated by applying a predetermined mathematical function.

According to an exemplary embodiment, the calibration displacement value is predetermined for each calibration projection.

According to an exemplary embodiment, the calibration displacement value is based on previous calibration measurements.

Thus, a so to speak self-learning system is provided where already measured misalignments can be taken into account for further calibration projections. Hence, it is possible to adapt the calibration projections to expected spatial behaviour of the system.

According to an exemplary embodiment, the calibration displacement value reproduces a virtual misalignment between the emitting arrangement and the detection arrangement during the measurement projections.

Thereby it is possible to adapt the calibration projections to the expected or already measured misalignment of the system, such that typical misalignments resulting from certain types or materials for the construction can be considered. This further improves the accuracy and therefore reliability of the achieved object information.

According to an exemplary embodiment, the measurement increment, or measurement increment factor, is a constant value.

For example, the calibration displacement value is at least half of the measurement increment value.

According to an exemplary embodiment, the object information is provided for further steps such as an analysis or further measurement steps.

According to an exemplary embodiment, the object information is displayed to the user on a display.

According to an exemplary embodiment, absorption rates are detected by the detector and the object information comprises absorption data, too.

According to an exemplary embodiment, the calibration displacement value is recorded for each of the calibration projections, and during the step c) of performing the second plurality of measurement projections, after one or more measurement projections at least one of the calibration projections is associated and the respective calibration displacement value is determined as misalignment factor, and before proceeding with the second plurality of measurement projections, the at least two actuators are activated such to realign the X-ray emitting arrangement with the phase-shift grating and the phase analyzer grating as well as the image detector.

This provides an alignment during the measurement scan process, for example during the examination of a patient. Thus, a so to speak live re-alignment is provided leading to a high accuracy of the results.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

It has to be noted that exemplary embodiments of the invention are described with reference to different subject matters. In particular, some exemplary embodiments are described with reference to apparatus type claims whereas other exemplary embodiments are described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

### BRIEF DESCRIPTION OF THE DRAWINGS

The aspect defined above and further aspects, features and advantages of the present invention can also be derived from the examples of embodiments to be described herein after and are explained with reference to examples of embodiments, but to which the invention is not limited. The invention will be described in more detail hereinafter with reference to the drawings.

FIG. 1 schematically shows an X-ray imaging system for differential phase-contrast imaging of an object according to the invention;

FIG. 2 schematically shows an X-ray emitting arrangement and an X-ray detection arrangement according to the invention;

FIG. 3 schematically shows the arrangement of FIG. 2;

FIG. 4 schematically shows gratings of the detection arrangement of FIG. 3;

FIG. 5 schematically shows the basic method steps according to an exemplary embodiment of the invention;

FIG. 6 shows another embodiment of the method;

FIG. 7 shows a further embodiment of the method; and

FIG. 8 schematically shows further steps of a further exemplary embodiment.

### DETAILED DESCRIPTION OF EXEMPLARY EMBODIMENTS

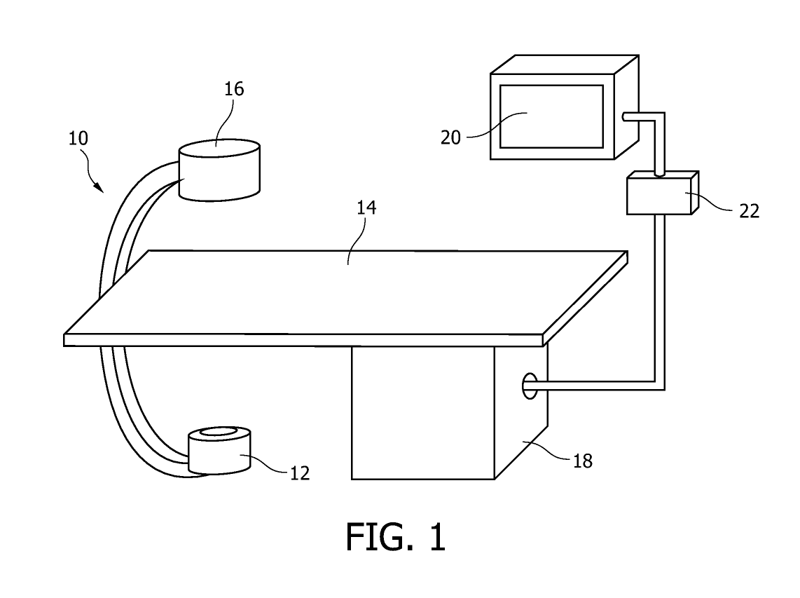

FIG. 1 schematically shows an X-ray imaging system 10 for differential phase-contrast imaging of an object, for example for the use in an examination laboratory, for example in a hospital. The X-ray imaging system comprises an X-ray emitting arrangement 12 adapted to provide at least partial coherent X-ray radiation. A table 14 is provided to receive a subject to be examined. Further, an X-ray detection arrangement 16 is located opposite the X-ray emitting arrangement 12, i.e., during the radiation procedure, the subject is located between the X-ray emitting arrangement 12 and the X-ray detection arrangement 16. The latter is sending data to a data processing unit 18 which is connected to both the X-ray detection arrangement 16 and the X-ray emitting arrangement 12. The processing unit 18 is located underneath the table 14 to safe space within the laboratory. Of course, it can also be located at a different place, such as a different room. Furthermore, a display device 20 is arranged in the vicinity of the table 14 to display information to the person operating the X-ray imaging system, for example a clinician such as a surgeon. Preferably the display device 20 is movably mounted to allow for an individual adjustment depending on the examination situation. Also, an interface unit 22 is arranged to input information by the user. Basically, the X-ray detection arrangement 16 generates images by exposing the subject to X-ray radiation, wherein said images are further processed in the data processing unit 18. It is noted that the example shown is of a so-called C-type X-ray image acquisition device. Of course, the invention also relates to other types of X-ray image acquisition devices, such as CT gantries or the like. The invention also relates to X-ray image acquisition devices, where the patient is arranged in a standing manner instead of lying on a table 14, such as acquisition devices for mammography and tomosynthesis. The X-ray emitting arrangement 12 and the X-ray detection arrangement 16 are described in more detail below.

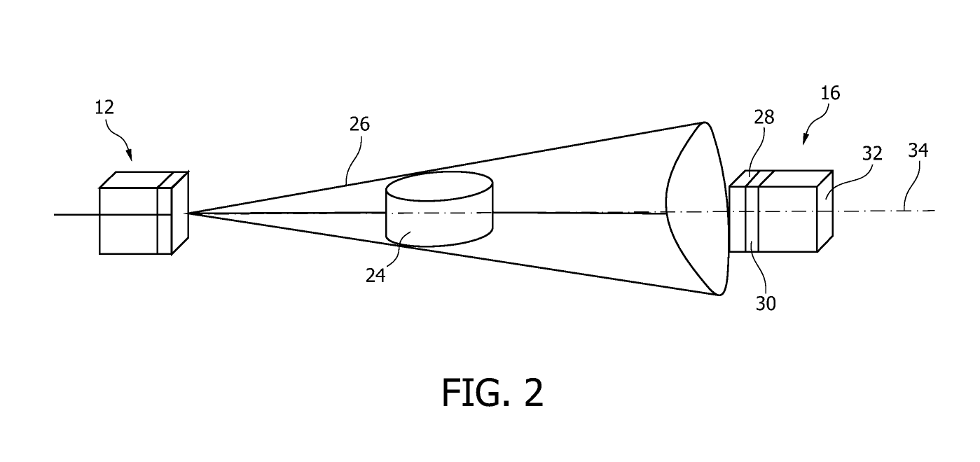

For a better understanding, FIG. 2 shows the X-ray emitting arrangement 12 and the X-ray detection arrangement 16 with an object 24 arranged between them. The table 14 of FIG. 1 as well as the display device 20 etc. are not shown in FIG. 2.

The X-ray emitting arrangement 12 provides at least partial coherent X-ray radiation 26. For example, the X-ray radiation comprises at least 20% coherent radiation. Preferably the radiation is 50% coherent.

According to an embodiment not shown, the X-ray emitting arrangement provides spatially coherent X-ray radiation.

The X-ray detection arrangement 16 comprises a phase-shift diffraction grating 28, a phase analyzer grating 30 and an X-ray image detector 32.

The X-ray emitting arrangement 12, the phase-shift grating 28 and the phase analyzer grating 30 and the image detector 32 are arranged in this order along an optical axis 34.

Further, for example, the phase-shift grating 28 and the phase analyzer grating 30 are arranged in planes parallel to each other.

The object 24 is receivable between the X-ray emitting arrangement 12 and the phase analyzer grating 30 such that a region of interest of the object is exposable to the X-ray radiation 26 emitting from the X-ray emitting arrangement 12 towards the detector 32.

According to one example, the object 24 is receivable between the X-ray emitting arrangement 12 and the phase-shift diffraction grating 28.

According to another example, not shown, the object 24 is receivable between the X-ray emitting arrangement 12 and the phase analyzer grating 30, i.e. in direction of the X-ray beams behind the phase-shift grating 28, or, in other words, between phase-shift grating 28 and the analyzer grating 30, such that a region of interest of the object is exposable to the X-ray radiation 26 emitting from the X-ray emitting arrangement 12 towards the detector 32.

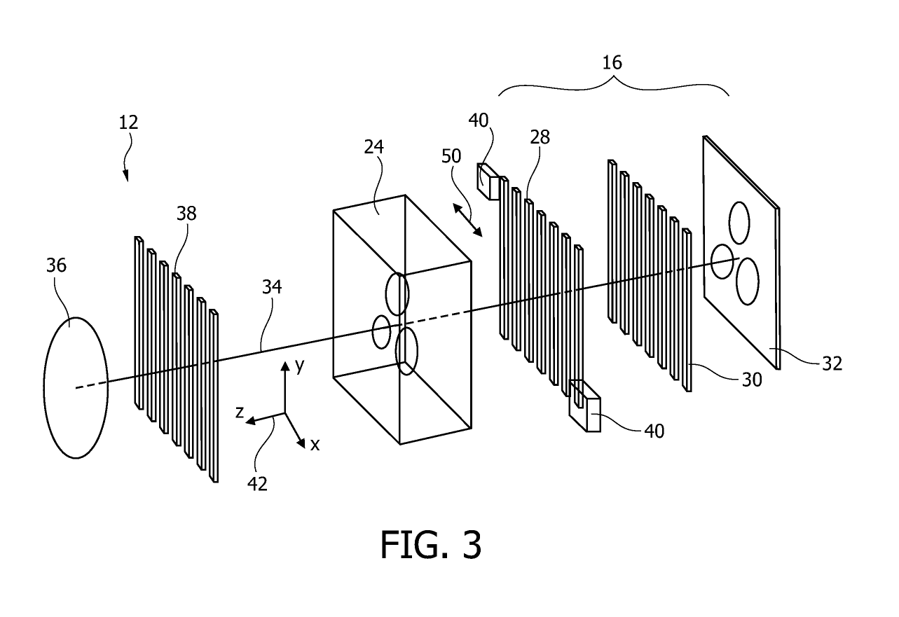

According to the invention, at least one of the group of one of the gratings 28, 30 and the X-ray emitting arrangement 12 is provided with at least two actuators arranged opposite to each other with reference to the optical axis 34, which actuators are not shown in FIG. 2, but will be explained with reference to FIG. 3.

As an exemplary embodiment, FIG. 3 shows a similar arrangement of the exemplary embodiment of FIG. 2, where for a better understanding, the X-ray detection arrangement 16 and also the X-ray emitting arrangement 12 are shown with their components spaced apart from each other.

In the embodiment of FIG. 3, the X-ray emitting arrangement 12 comprises an X-ray source 36 emitting incoherent X-ray radiation and a source grating 38 is placed close to the X-ray source 36 to provide spatial beam coherence in order to provide the above-mentioned at least partially coherent X-ray radiation 26. The phase-shift diffraction grating 28 is provided with two actuators 40 arranged opposite to each other with reference to the optical axis 34. As an example, the gratings 38, 28, 30 are rectangular and the actuators 40 are arranged diametrically to each other.

In a further embodiment, not shown, the X-ray emitting arrangement 12 comprises an X-ray source emitting at least partially coherent X-ray radiation, for example by providing a micro-focus tube or a synchrotron-type tube as X-ray source. In a further example, carbon nano-tubes are provided to generate the at least partial coherent X-ray radiation.

As indicated by coordinate system 42, the optical axis is referred to as the z-axis, the grid orientation which is perpendicular to the z-axis, is referred to as the y-axis and the axis perpendicular to the grid orientation and perpendicular to the optical axis is referred to as the x-axis.

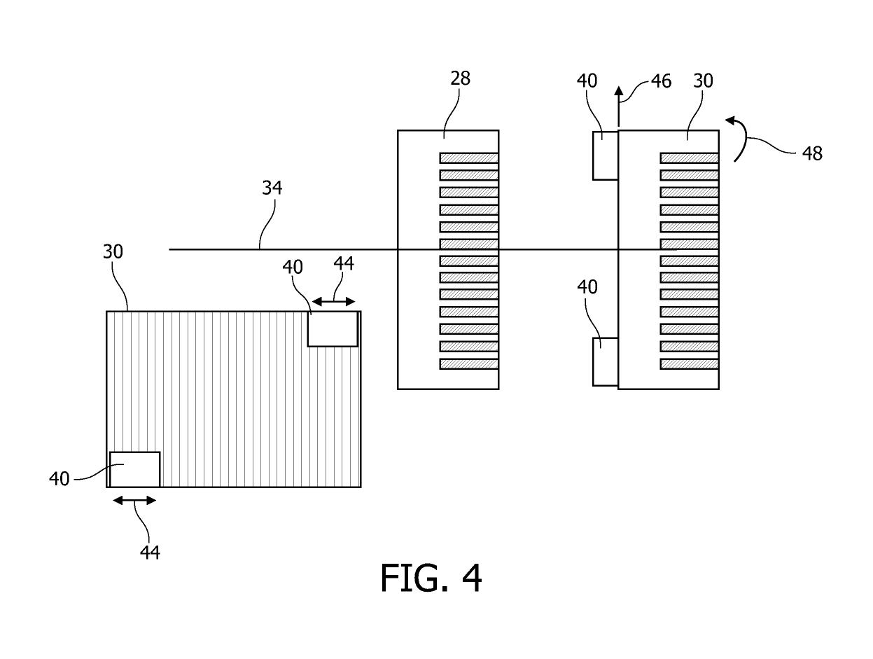

As can be seen from FIG. 3, the actuators 40 form a double actuator, which will be explained in the following. The two actuators 40 each provide linear movement in a direction which is perpendicular to the grid orientation and which is also perpendicular to the optical axis 34. In other words, the actuators 40 provide movement in the x-axis, as indicated by arrows 44 in FIG. 4.

In FIG. 4, the phase analyzer grating 30 is provided with actuators 40 instead of the phase-shift diffraction grating 28, as this is shown in FIG. 3.

FIG. 4, in the lower left part shows a view of the phase analyzer grating 30 in the direction of the optical axis 34 and the upper right part shows the phase-shift diffraction grating 28 and the phase analyzer grating 30 in a so to speak top view. As indicated by arrow 46, the at least two actuators 40 providing movement 44 in the x-axis provide for linear movement, indicated by arrow 46, of the grating by moving of the actuator 40 with same speed in same direction.

By moving the actuators 40 in different directions, rotational movement is provided indicated by arrow 48. Of course, this rotational movement depends on the fixture point of the grating.

According to the invention, the at least two actuators 40 provide stepping movement of at least one of group of one of the gratings 28, 30 and the X-ray emitting arrangement 12 for phase stepping image acquisition and calibration movement for calibrating the system in order to detect and to compensate misalignment of the X-ray emitting arrangement 12 and the phase-shift grating 28 and the phase analyzer grating 30.

According to another exemplary embodiment, the source grating 38 is provided with two actuators (not shown).

The two actuators 40 are provided as piezo-drive elements, for example, with a solid-state hinge. For example, the actuators 40 are integrally implemented with the grating, i.e., the source grating 38, the phase-shift diffraction grating 28 or the phase analyzer grating 30, in silicon, for example. According to a further exemplary embodiment, which is not shown, at least one additional actuator is provided which actuator is adapted for movement in the direction of the optical axis 34 such that at least one of the gratings can be tilted in relation to the optical axis.

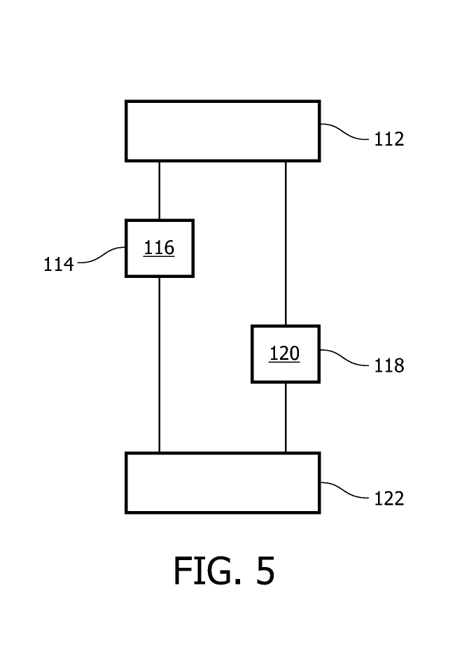

According to an exemplary embodiment, a method for acquisition of information about an object is provided, which will be explained with reference to FIG. 5. At least partially coherent X-ray radiation is emitted 112 from the X-ray emitting arrangement 12 towards an X-ray detection arrangement 16. The X-ray detection arrangement 16 comprises a phase-shift diffraction grating 28, the phase analyzer grating 30 and the X-ray image detector 32. The X-ray emitting arrangement 12, the phase-shift grating 28, the phase analyzer grating 30 and the image detector 32 are arranged along the optical axis 34.

Further, as an example, the phase-shift grating 28 and the phase analyzer grating 30 are arranged in planes parallel to each other.

The emitted coherent X-ray radiation 26, the phase-shift grating 28 and the phase analyzer grating 30 have a common grid orientation, for example the y-axis of the coordinate system 42. In a first performing step 114, a first plurality of calibration projections 116 is performed without an object. During the first plurality of calibration projections 116, the emitted X-ray radiation 26 or one of the group of the phase-shift grating 28 and the phase analyzer grating 30 is stepwise displaced during this performance of the calibration projections with a calibration displacement value, indicated by arrow 50 in FIG. 3.

For example, the displacement comprises translation, rotation, and tilting of the gratings. The term “stepwise displacement” comprises a one-dimensional movement as well as a two- or more-dimensional movement, e.g. a three-dimensional movement track in space. Thus it is possible to create a multidimensional parameter space, or multidimensional movement space. Thereby, the calibration projections can be adapted to different possible misalignments. As an example, the displacement value is a predetermined factor with same value for each step. Alternatively, the displacement value changes constantly, for example by a constant mathematical function or by predetermined fixed values.

Further, in a second performance step 118, a second plurality of measurement projections 120 is performed with an object arranged between the X-ray emitting arrangement 12 and the phase analyzer grating 30. During the second plurality of measurement projections 120, the emitted X-ray radiation 12, or one of the group of the phase-shift grating 28 and the phase analyzer grating 30 is stepwise displaced with a measurement increment. The calibration displacement value differs from the measurement increment, which will be described further below.

For example, the object is arranged between the X-ray emitting arrangement 12 and the phase-shift diffraction grating 28.

According to another example, not shown, the object is arranged between phase-shift grating 28 and the analyzer grating 30.

For example, the stepwise displacement during the measurement projections is provided as a stepwise movement perpendicular to the grid orientation.

In an associating step 122, at least one of the calibration projections 116 is associated to each of the measurement projections 120 by registering the measurement projections 120 with a calibration scan 116.

For example, in order to register the calibration projection with the measurement projection, the measurement projection is analyzed for parts which are illuminated directly. Depending on the actual position of the gratings, for example due to translation, rotation, tilt or the like, a characteristic fringe pattern is visible in these areas. In the second step of the registration process, the projection from the plurality of the calibration projections is identified which shows in the same area the most similar fringe pattern.

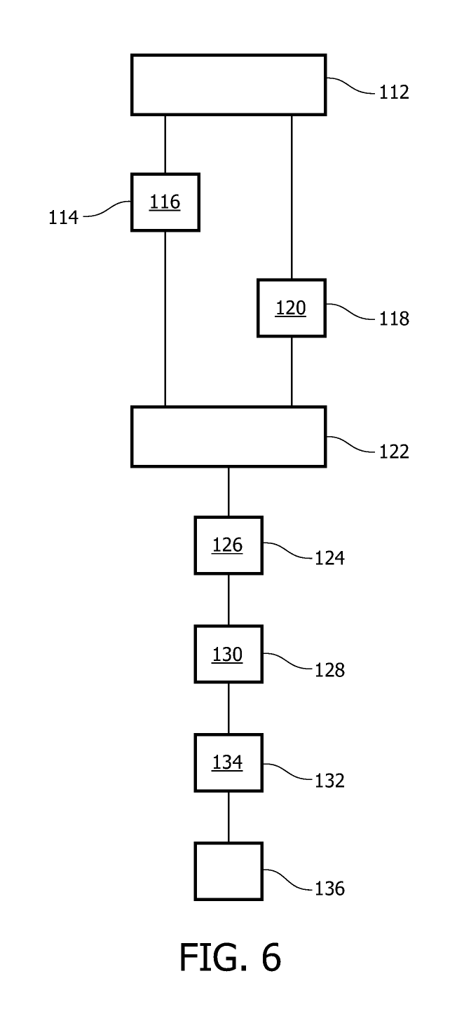

According to one exemplary embodiment shown in FIG. 6, in a generating step 124, adjusted measurement projections 126 are generated by subtracting the respective associated calibration scan 116 from each of the measurement projections 120.

In a determination step 128, differential phase data 130 is determined from the adjusted measurement projections 126. Next, in a generating step 132, object information 134 is generated on behalf of the determined differential phase data 130.

According to an embodiment, the object information 134 is provided.

For example, the object information is displayed to the user 136 on a display.

The displacement during the calibration projections 116 and the displacement during the measurement projections 120 are provided by the actuators 40 described above.

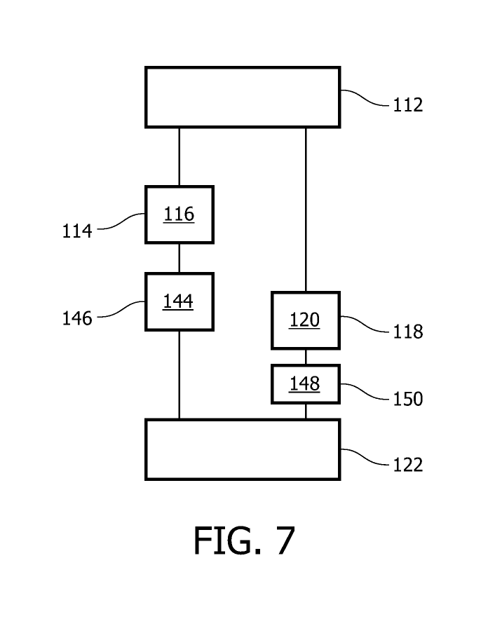

According to another exemplary embodiment, shown in FIG. 7, after the first performance step 114, phase gradient data 144 is determined 146 for each of the calibration projections 116 and after the second performance step 118 phase gradient data 148 is determined 150 for each of the measurement projections 120.

According to a further embodiment, the misalignment of the system is detected. Therefore, the calibration displacement value is recorded for each of the calibration projections. This factor represents a sort of virtual misalignment of the system. This information can then be used to determine the actual or real misalignment during the measurement projections. The result, i.e., the real misalignment factors can be used to adapt the calibration displacement value values for further projections. In other words, the calibration displacement value is based on previous calibration measurements. This provides a self-learning system where already measured misalignments can be taken into account for further calibration projections. Hence, it is possible to adapt the calibration projections to expected spatial behaviour of the system. For example, certain type of measurement projections will have certain misalignment profiles, for example, due to constructional aspects. For example, during C-arm investigations, certain bending or twisting will occur in the same positions. As another example, in breast cancer examinations, the paddle holding the breast will lead to the same type of bending forces leading to similar misalignments.

In a further exemplary embodiment not shown, the X-ray emitting arrangement 12 comprises the X-ray source 36 emitting incoherent X-ray radiation and the source grating 38 is placed close to the X-ray source 36 to provide spatial beam coherence. The source grating is displaced during the calibration projections 116 and during the measurement projections 120.

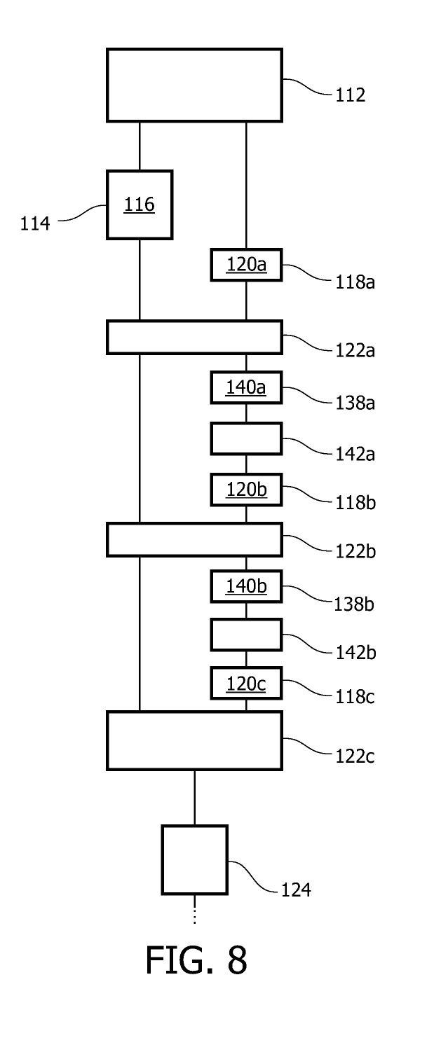

According to a further exemplary embodiment of the invention, shown in FIG. 8, the calibration displacement value is recorded for each of the calibration projections 116. During the performance step 118 of performing the second plurality of measurement projections 120, after one or more measurement projections 120a at least one of the calibration projections 126 is associated 122a and the respective calibration displacement value is determined 138a as misalignment factor 140a. Before proceeding with the second plurality of measurement projections 120b, the at least two actuators 40 are activated such to realign 142a the X-ray emitting arrangement 12 with the phase-shift grating 28 and the phase analyzer grating 30 as well as the image detector 32. Next, the second plurality of measurement projections is performed in a further performance step 118b leading to measurement projections 120b. Following, in a further associating step 122b, the acquired measurement projections 120b are associated 122b to at least one of the calibration projections 116 and the respective calibration displacement value is determined 138b as misalignment factor 140b for a further realignment step 142b before further measurement projections 120c are acquired in a further part of the performance step, i.e., for example in a third performance step 118c.

This is followed by a further associating step 122c which can be repeated depending on the needs.

This is then followed by the generation step 124 following as described above.

In other words, even during a measurement procedure, it is possible to re-align the system in order to improve the quality and exactness of the generated object or patient information. Thus, the invention provides a live-alignment or alignment in real time.

It is noted that the embodiments of the method steps shown in FIG. 5, FIG. 6, FIG. 7 and FIG. 8 can be combined with each other in different combinations.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage.

A computer program may be stored and/or distributed on a suitable medium, such as an optical storage medium or a solid state medium supplied together with or as part of other hardware, but may also be distributed in other forms, such as via the internet or other wired or wireless telecommunication systems.

Any reference signs in the claims should not be construed as limiting the scope.

1. X-ray imaging system for differential phase contrast imaging of an object, comprising:
an X-ray emitting arrangement (12); and
an X-ray detection arrangement (16);
wherein the X-ray emitting arrangement (12) provides at least partially coherent X-ray radiation (26);
wherein the X-ray detection arrangement (16) comprises:
a phase-shift diffraction grating (28);
a phase analyzer grating (30); and
an X-ray image detector (32);
wherein the X-ray emitting arrangement (12), the phase-shift grating (28) and the phase analyzer grating (30) and the image detector (32) are arranged in this order along an optical axis (34);
wherein an object to be examined is receivable between the X-ray emitting arrangement (12) and the phase analyzer grating (30) such that a region of interest of the object is exposable to X-ray radiation emitting from the X-ray emitting arrangement (12) towards the detector (32); and
wherein at least one of the group of one of the gratings (28, 30) and the X-ray emitting arrangement (12) is provided with at least two actuators (40) arranged opposite to each other with reference to the optical axis (34).

2. X-ray imaging system according to claim 1, wherein the at least two actuators (40) provide stepping movement of at least one of the group of one of the gratings (28, 30) and the X-ray emitting arrangement (12) for phase stepping image acquisition and calibration movement for calibrating the system in order to detect and to compensate misalignment of the X-ray emitting arrangement (12) and the phase-shift grating (28) and the phase analyzer grating (30).

3. X-ray imaging system according to claim 1, wherein the X-ray emitting arrangement (12) comprises an X-ray source (36) emitting incoherent X-ray radiation; and wherein a source grating (38) is placed close to the X-ray source (36) to provide spatial beam coherence.

4. X-ray imaging system according to claim 1, wherein the at least two actuators (40) each provide linear movement in a direction which is perpendicular to the grid orientation and which is also perpendicular to the optical axis (34).

5. X-ray imaging system according to claim 1, wherein the at least two actuators (40) are provided as piezo drive elements with a solid state hinge.

6. Method for acquisition of information about an object, comprising the following steps:
a) emitting (112) at least partially coherent X-ray radiation (26) from an X-ray emitting arrangement (12) towards an X-ray detection arrangement (16);
wherein the X-ray detection arrangement (16) comprises a phase-shift diffraction grating (28), a phase analyzer grating (30) and an X-ray image detector (32);
wherein the X-ray emitting arrangement (12), the phase-shift grating (28), the phase analyzer grating (30) and the image detector (32) are arranged along an optical axis (34); and
wherein the emitted at least partially coherent X-ray radiation, the phase-shift grating (28) and the phase analyzer grating (30) have a common grid orientation;
b) performing (114) a first plurality of calibration projections (116) without an object;
wherein, during the first plurality of calibration projections, the emitted X-ray radiation or one of the group of the phase-shift grating (28) and the phase analyzer grating (30) is stepwise displaced with a calibration displacement value;
c) performing (118) a second plurality of measurement projections (120) with an object (24) arranged between the X-ray emitting arrangement (12) and the phase analyzer grating (30);
wherein, during the second plurality of measurement projections, the emitted X-ray radiation (26) or one of the group of the phase-shift grating (28) and the phase analyzer grating (30) is stepwise displaced with a measurement increment; and
d) associating (122) at least one of the calibration projections (116) to each of the measurement projections (120) by registering the measurement projections (120) with the calibration projections (116).

7. Method according to claim 6, wherein after step d) the following steps are performed:
e) generating (124) adjusted measurement projections (126) by subtracting the respective associated calibration scan (116) from each of the measurement projections (120);
f) determining (128) differential phase data (130) from the adjusted measurement projections (126); and
g) generating (132) object information (134) on behalf of the determined differential phase data (130).

8. Method according to claim 6, wherein following step b) phase gradient data (144) is determined (146) for each of the calibration projections (116); and wherein following step c) phase gradient data (148) is determined (150) for each of the measurement projections (120).

9. Method according to claim 6, wherein the X-ray emitting arrangement (12) comprises an X-ray source (36) emitting incoherent X-ray radiation and a source grating (38) is placed close to the X-ray source (36) to provide spatial beam coherence;
wherein the source grating (38) is displaced during the calibration projections (116) and during the measurement projections (120).

10. Method according to claim 6, wherein at least one of the group of one of the gratings (28, 30; 38) and the X-ray emitting arrangement (12) is provided with at least two actuators (40) arranged at the grating opposite to each other with reference to the optical axis (34); wherein the at least two actuators (40) provide the displacement during the calibration projections (116) and during the measurement projections (120).

11. Method according to claim 6, wherein the calibration stepwise displacement comprises a stepping in a direction perpendicular to the grid orientation.

12. Method according to claim 6, wherein the calibration displacement value is recorded for each of the calibration projections (116); and wherein during the step c) of performing (118a, 118b, 118c) the second plurality of measurement projections (120a, 120b, 120c), after one or more measurement projections at least one of the calibration projections (116) is associated (122a, 122a, 122c) and the respective calibration displacement value is determined (138a, 138b, 138c) as misalignment factor (140a, 140b, 140c); and before proceeding with the second plurality of measurement projections, the at least two actuators (40) are activated such to re-align (142) the X-ray emitting arrangement (12) with the phase-shift grating (28) and the phase analyzer grating (30) as well as the image detector (32).

13. Computer program element for controlling an apparatus according to claim 1.

14. Computer readable medium having stored the program element of claim 13.
